(19) United States
(12) Patent Application Publication
(10) Pub. No.: US 2010/0303745 A1
(43) Pub. Date: Dec. 2, 2010

(54) SKIN BRONZER

(75) Inventors: Meredith Fish, New York, NY (US); Pauley Fei-Zan Chang, Diamond Bar, CA (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: Brownberry, New York, NY (US)

(21) Appl. No.: 12/689,519

(22) Filed: Jan. 19, 2010

Related U.S. Application Data
(60) Provisional application No. 61/182,688, filed on May 29, 2009.

(51) Int. Cl. A61K 8/35

(52) U.S. Cl. 424/60; 424/59

(57) ABSTRACT

The invention provides a composition having a sunblock and tanning effect containing dihydroxyacetone and a broad spectrum UV blocker having a SPF of at least about 25.

### PRIORITY CLAIM

This application claims priority to application Ser. No. 61/182,688, filed May 30, 2009 under 35 USC §119(e), the contents of which are incorporated herein by reference.

### FIELD OF THE INVENTION

The invention provides a composition containing dihydroxyacetone and a broad spectrum UV blocker having a SPF of at least about 25.

### BACKGROUND OF THE INVENTION

The harmful effect of the ultraviolet part of solar radiation on the skin is generally known. In addition, sunbathing and/or exposure to sun also lead to a drying-out of the skin. This drying-out can be superficial in nature and predominantly affect the horny layer. However, it can also have a type of “deep drying-out” effect on the living cell layers of the skin. The extent of surface drying can be determined, for example, using corneometry. The extent of deep drying (the reduction in deep moisture), by contrast, can be ascertained, for example, by cell size and/or cell volume determinations of living cells in the deeper skin layers, in particular the living cells of the epidermis. However, a healthy (natural) tan is viewed by the consumer as being desirable. Of importance there is not only a general “somehow darker” complexion, but rather a sporty, vital and fresh (natural) skin tanning, as is gained by gradual (gentle) sunbathing, which does not lead to sunburn. The skin tanning which arises, for example, after a sunbed session is, by contrast, regarded as being unnatural or grayish. The color tone which can be achieved with self-tanning compositions as sole pigmentation carriers also barely corresponds to the color of natural healthy skin.

Therefore, there is a need for a composition that will allow for the appearance of a “healthy tan” but will protect the skin from deleterious effects of ultraviolet light. There is also a need for a composition that provides a flawless rub-free application.

Furthermore, the invention includes aerosolized formulation providing a flawless, rub free application, and results in a more effective and uniform protection and tanning.

### SUMMARY OF THE INVENTION

The invention is directed to a composition/formulation/preparation having at least about 25 SPF and preferably about 30 SPF comprising (a) a self-tanning formulation comprising dihydroxyacetone and optionally troxerutin and (b) one or more ultraviolet blockers. In a particular embodiment, the composition is a water resistant spray and an aerosolized formulation. In a more particular embodiment, the invention is directed to a composition having an SPF of at least about 30 comprising (a) about 1-15 wt. % dihydroxyacetone; (b) about 1-15 wt. % dihydroxyacetone/troxerutin formulation; (c) about 1-3 wt. % avobenzone and/or about 7-15% homosalate; and (d) at least one of: about 0.2-5 wt. % of one or more emollients; about 5-20% wt. % of one or more emulsifiers; about 1-8 wt. % of one or more solubilizers and/or about 0.5-2 wt. % of thickeners. In yet another particular embodiment, the invention relates to a composition having an SPF of at least about 25 in which the active ingredients consist essentially of (a) dihydroxyacetone and/or dihydroxyacetone/troxerutin formulation and (b) one or more UV protection agents.

The invention is further directed to methods of using this composition, particularly a method for artificially tanning skin, method for increasing or improving the natural endogenous UV protection of skin, a method for achieving a natural skin shade during or after exposure to sunlight or UV light, all of which involve applying to the skin an amount of the composition effective for artificially tanning skin, increasing or improving the natural endogenous UV protection of skin, or achieving a natural skin shade during or after exposure to sunlight or UV light. In a related aspect, the invention is directed to use of or using dihydroxyacetone and one or more UV blockers to make the composition of the present invention for the uses set forth above.

### DETAILED DESCRIPTION OF THE INVENTION

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

The terms “preparation”, “composition” and “formulation” are used interchangeably.

As noted above, the invention is directed to a preparation, composition or formulation comprising a self tanning preparation comprising dihydroxyacetone and optionally troxretutin and one or more UV blockers with a sun protection factor of at least about 25. The value of the SPF (Sun Protection Factor, also called LPF for Light Protection Factor) is determined in vivo in accordance with COLIPA. In a more particular embodiment, the preparation has an SPF of at least about 30.

Dihydroxyacetone, may also be referred to as DHA or 1,3-Dihydroxypropan-2-on. In another embodiment, the composition may further comprise a second self tanning agent such as troxerutin, which may also be referred to as hydroxyethylrutoside (HER), Pherarutin Trihydroxyethylrutin 3′,4′,7-Tris[0-(2-hydroxyethyl)]rutin. In a particular embodiment, the ratio of troxerutin to dihydroxyacetone is from about 30:1 to about 1:1

Preferably the composition is an aqueous formulation but is water resistant (does not wash off when exposed to water). In particular, the composition comprises about 1 to 15 wt. % dihydroxyacetone and/or troxerutin, and more particularly about 1-6 wt. % of dihydroxyacetone, and/or troxerutin and about 12 to 33 wt. % of one or more UV blocking agents. In a specific embodiment, the composition comprises both an agent that blocks the UVB portion of the UV spectrum and an agent that blocks the UVA portion of the UV spectrum. The UVB portion encompasses 280-320 nm and the UVA portion encompasses 320-400 nm. The UV blocking agent includes but is not limited to avobenzone, and homosalate, and/or octocrylene, oxybenzone, octisalate, octinoxate, zinc oxide, 2-phenylbenzimididazole-5-sulfonic acid, methyl-O-aminobenzoate. In a specific embodiment, homosalate is present in the amount of about 7-15 wt. % and avobenzone is present in the amount of about 1.5-3.0 wt. %.

The composition may further comprise other ingredients. A further ingredient of the composition of the present invention is an emollient. As defined herein, an “emollient” is a material that protects against wetness or irritation, softens, soothes, supples, coats, lubricates, moisturizes, protects and/or cleanses the skin. Examples include but are not limited to aloe vera gel, caprylyl glycol, dipotassium glycyrrhizinate, avena sativa (Oat) kernel extract and glycerin. Emollients may be present in the amount of about 0.2-5 wt. %. The composition of the present invention may also comprise emulsifiers in an amount of about 5-20 wt. % including but not limited to steareth-100 (and) steareth-2, PEG-25 hydrogenated castor oil, PEG-40 hydrogenated castor oil acrylates/C12-22 alkylmethacrylate copolymer and/or acrylates copolymer and/or solubilizers (about 1-8 wt. %), and/or thickeners present in the amount of about 0.5-about 2.0 wt. % such as mannan and xanthan gum In a specific embodiment the preparation or composition comprises DHA/troxerutin (about 1-6 wt. %) and homosalate (about 7-15 wt. %) and/or avobenzone (about 1-3 wt. %) as the primary active ingredients, about 1-3 wt. % of diethylhexyl syringal malonate/caprylic/capric triglyceride and about 1-8 wt. % of penethyl benzoate as the primary solubilizers hexylene glycol, PEG-25 hydrogenated castor oil, PEG-40 hydrogenated castor oil (2-8 wt. %) as the key emulsifiers along with acrylates/C12-22 alkylmethacrylate copolymer (1-3 wt. %) and/or acrylates copolymer (2-6 wt. %) in this invention.

The compositions of the present invention may be applied to the skin in the amount of about 0.1-10 mg/cm² of skin. The composition of the present invention would be applied as needed and may be applied throughout the day.

### Example

The example below is presented to describe preferred embodiments and utilities of the invention and is not meant to limit the invention unless otherwise stated in the claims appended hereto.

Table I below shows one particular composition of the present invention.

TABLE I

| Phase | DESCRIPTION | WT. % |
|---|---|---|
| A | Purified Water | 27.2500 |
| A | Steareth-100 (and) Steareth-2 (and) Mannan | 1.0000 |
| | (and) Xanthan Gum | |
| A | Glycerin | 2.1000 |
| B | Homosalate | 15.0000 |
| B | Diethylhexyl Syringal Malonate/ | 2.5000 |
| | Caprylic/Capric Triglyceride | |
| B | Penethyl Benzoate | 3.0000 |
| B | Hexylene glycol, PEG-25 hydrogenated | 5.0000 |
| | castor oil, PEG-40 hydrogenated castor oil | |
| B | Avobenzone | 2.5000 |
| B | (PEG-12 Dimethicone) | 0.2500 |
| B | (Caprylyl Glycol) | 1.0000 |
| C | Purified Water | 20.0000 |
| C | Dihydroxyacetone | 12.0000 |
| C | (Dihydroxyacetone,/Troxerutin) | 2.0000 |
| C | Dipotassium glycyrrhizinate | 0.3000 |
| C | Water, glycerin avena sativa kernal extract | 2.0000 |
| C | Aloe Con UP 10 COS | 0.5000 |
| D | (Acrylates/C12-22 Alkylmethacrylate | 2.1000 |
| | Copolymer) | |
| E | Phenoxyethanol | 1.0000 |
| F | Fragrance-Naturally Green R08-0672 | 0.5000 |

The following procedure is used to obtain the above-referenced composition.

Water is discharged to a clean, sanitized tank. Steareth-100 (and) Steareth-2 (and) Mannan (and) Xanthan Gum is added while mixing. The mixture is heated to 80 C and mixed until homogeneous (20 minutes after reaching 80 C), add glycerin, mix until uniform. Cool to 70 C. Homosalate, Finsolv Sun and Hexylene glycol, PEG-25 hydrogenated castor oil, PEG-40 hydrogenated castor oil is added. The mixture is heated to 60-70 C and Lexegard 0 and Avobenzone is added and mixed until uniform. Silsoft 900 is added and mixed in until homogeneous & transparent. Phase (B) and (A) are added to the Main Tank while both phases are @ 70 C and are mixed until uniform. Premix Phase (C) is premixed by adding ingredients in the order listed and then mixed until uniform between addition Acrylates/C12-22 Alkylmethacrylate Copolymer is added to the main tank and mixed for 30 minutes. The mixture is cooled to 30-35 C. Phenoxyethanol is added to the main tank and mixed for 10 minutes. Fragrance is added and mixed for 20 minutes with low speed throughout the production.

The aerosol formulation is to fill 20 to 80 wt. % by weight of the above liquid bulk and pressurized with 20 to 80 wt. % of Dimethyl Ether and/or 0 to 40 wt. % of 1,1 Difluoroethane as the propellant(s) in an aluminum container with PAM (Polyamide-imide resin) lining (Valve, actuator and other packaging component details to follow if needed.)

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosures of which are incorporated by reference in their entireties.

What is claimed is:

1. A composition having an SPF of at least about 25 comprising (a) a self-tanning formulation comprising dihydroxyacetone and optionally troxerutin and (b) one or more UV protection agents.

2. The composition according to claim 1 wherein said composition has an SPF of at least about 30.

3. The composition according to claim 1, wherein said composition is a water resistant composition.

4. The composition according to claim 1, wherein said composition is a spray composition including aerosol formulation.

5. The composition according to claim 1, wherein said UV protection agent is avobenzone, and homosalate.

6. The composition according to claim 1, wherein said composition further comprises one or more emollients, humectants, thickeners and/or antioxidants.

7. The composition according to claim 1, wherein said composition comprises about 1 wt. % to 15 wt. % of dihydroxyacetone and about 12-33 wt. % of one or more UV protection agents.

8. The composition according to claim 1, wherein the self-tanning formulation comprises dihydroxyacetone and troxerutin, wherein the ratio of troxerutin:dihydroxyacetone is about 30:1 to about 1:1.

9. A composition having an SPF of at least about 30 comprising (a) about 1-15 wt. % dihydroxyacetone; (b) about 1-15 wt. % dihydroxyacetone/troxerutin formulation; (c) about 1-3 wt. % avobenzone and/or about 7-15% homosalate; and (d) at least one of: about 0.2-5 wt. % of one or more emollients; about 5-20% wt. % of one or more emulsifiers; about 1-8 wt. % of one or more solubilizers and/or about 0.5-2% of thickeners.

10. A method for artificially tanning skin, increasing or improving the natural endogenous UV protection of skin and/or achieving a natural skin shade during or after exposure to sunlight or UV light, comprising applying to the skin an amount of the composition according to claim 1 effective for artificially tanning skin, increasing or improving the natural endogenous UV protection of skin, or achieving a natural skin shade during or after exposure to sunlight or UV light.

11. A method for artificially tanning skin, increasing or improving the natural endogenous UV protection of skin and/or achieving a natural skin shade during or after exposure to sunlight or UV light, comprising applying to the skin an amount of the composition according to claim 8 effective for artificially tanning skin, increasing or improving the natural endogenous UV protection of skin, or achieving a natural skin shade during or after exposure to sunlight or UV light.

12. A composition having an SPF of at least about 25 in which the active ingredients consist essentially of (a) dihydroxyacetone and/or dihydroxyacetone/troxerutin formulation and (b) one or more UV protection agents.
